(19) United States
(12) Patent Application Publication
(10) Pub. No.: US 2012/0329749 A1
(43) Pub. Date: Dec. 27, 2012

(54) Methods Of Preventing Or Treating Disease States Related To Certain Metabolic Abnormalities

(76) Inventors: Stephen Albert Lonnie Smith, Alexandria, LA (US); Jeffrey Lynn Barringer, Lowell, AR (US); Craig Jay Brown, Fayetteville, AR (US); Gilberto Sanchez, Cecil, AL (US); Stephanie Michelle Ott, Prattville, AL (US); Brian Buell, Fayetteville, AR (US)

(21) Appl. No.: 13/528,393

(22) Filed: Jun. 20, 2012

Related U.S. Application Data
(60) Provisional application No. 61/499,393, filed on Jun. 21, 2011.

(51) Int. Cl. A61K 31/714; A61P 3/06; A61P 25/00; A61P 25/06; A61P 21/00; A61P 3/00; A61P 15/10

(52) U.S. Cl. 514/52

(57) ABSTRACT

The present invention relates to methods of treating, preventing, and/or improving disorders and conditions related to certain metabolic abnormalities including methods of improving blood lipid profiles and muscle recovery and repair in athletes, methods of treating fibromyalgia, erectile dysfunction, diseases associated with certain HLA-DQ gene alleles and/or gluten intolerance, including Celiac Disease, headaches and migraines, as well as idiopathic neuropathy, inability to sleep, inability to concentrate, and/or neurological development issues in children, the methods involving the administration of one or more downstream folate compounds and optionally methyl-B12. In one particular embodiment, the method comprises administration of L-methylfolate. In certain embodiments, the method further involves identifying a subject organism with a malfunction in one or more of the folate or B4 cycles. In certain embodiments, such a malfunction is one or more of the C677T and A1298C genetic polymorphisms.

### FIELD OF THE INVENTION

The invention relates to methods of treating, preventing, and improving disorders and conditions related to certain metabolic abnormalities using metabolically active folate compounds, optionally in combination with methylcobalamin, including methods of improving blood lipid profiles and methods of treating fibromyalgia; erectile dysfunction; diseases associated with certain HLA-DQ gene alleles and/or gluten intolerance, including Celiac Disease; headaches and/or migraines; muscle recovery and/or repair in athletes; as well as idiopathic neuropathy, inability to sleep, inability to concentrate, and/or neurological development issues in children.

### BACKGROUND OF THE INVENTION

Coronary Heart Disease (CHD) is the leading cause of death in the United States today. (American Heart Association, Cardiovascular Disease Statistics, available at www.americanheart.org, accessed Oct. 11, 2010). It is estimated that over 1.25 million Americans will have a coronary attack in 2010 alone. CHD is most often caused by atherosclerosis, which involves the thickening of artery walls and subsequent restriction of blood flow as a result of the build-up of fatty materials in the arteries. Known risk factors for atherosclerosis include, but are not limited to, high serum cholesterol levels, high triglyceride levels, high levels of low-density lipoproteins (LDL), low levels of high-density lipoproteins (HDL), and a high LDL to HDL ratio.

Atherosclerosis often begins in early adolescence, and can remain asymptomatic for decades. As the complications of advanced atherosclerosis are slowly progressive, cumulative, and chronic, the first symptom is often heart attack. Consequently, it is imperative to carefully monitor and regulate the blood lipid profile in order to prevent over-accumulation and abnormal deposition of fatty materials within the body.

Cholesterol is a steroid metabolite present in the cell membranes and blood plasma of all animals. It is important for the production of bile acids and steroid hormones and for the absorption of fat-soluble vitamins, including vitamin A, vitamin D, vitamin E, and vitamin K. Serum cholesterol derives from two sources: food and endogenous biosynthesis. Dietary sources of cholesterol include foods containing animal fats, most notably, cheese, egg yolks, beef, pork, poultry, and shrimp, whereas endogenously synthesized cholesterol is produced primarily by the liver. Insoluble in the bloodstream, cholesterol is transported through the body by lipoproteins.

Two common types of lipoproteins involved in serum cholesterol transport are high-density lipoproteins and low-density lipoproteins. HDLs are known to remove excess cholesterol from tissues and transport it to the liver for excretion, whereas LDLs are known to deposit cholesterol on artery walls. High serum levels of LDL cholesterol are strongly associated with cardiovascular disease because they promote development of atheromatous plaques in arteries.

Triglycerides are the chemical form in which most fat exists in food and the human body. They are esters derived from glycerol and three fatty acids and are the main constituent of vegetable oil and animal fats. High serum levels of triglycerides have been linked to atherosclerosis, which is at least partially due to the strong inverse relationship between triglyceride levels and HDL cholesterol levels in the blood.

The Western diet is notoriously high in saturated and trans-fatty acids, and, as a result, over 100 million Americans have abnormal blood lipid profiles. (American Heart Association, Cholesterol Statistics, www.americanheart.org, accessed Sep. 30, 2010). Current commonly prescribed treatments to reduce or lower blood lipid levels include statins, bile-acid resins, fibric acid derivatives, cholesterol absorption inhibitors, and dietary changes directed at lowering overall trans-fatty acid and saturated fat intake. A link between metabolically active folate and/or methylcobalamin deficiency and blood lipid profiles that indicate an increased risk for CHD has not heretofore been established.

Fibromyalgia is a medical disorder characterized by chronic, widespread, musculoskeletal pain, accompanied by allodynia, fatigue, sleep, memory and mood issues. It is estimated that fibromyalgia affects about 2-4% of the general population with a female to male ration of about 9:1. (Bartels, E. M., et al., “Fibromyalgia, diagnosis and prevalence: Are gender differences explainable?”. Ugeskr Laeger. 171 (49): 3588-92 (2009)). Some studies further suggest that 20-30% of patients with rheumatoid arthritis may also have fibromyalgia. (Yunus, M. B., “Role of central sensitization in symptoms beyond muscle pain, and the evaluation of a patient with widespread pain.” Best Pract Res Clin Rheumatol, 21 (3): 481-97 (2007)). Fibromyalgia patients incur higher health care costs as currently available treatment options are usually paired with psychological counseling designed to help patients learn how to cope with pervasive lifelong pain. Despite widespread speculation, the cause of fibromyalgia is currently unknown. Moreover, to the best of the inventors' knowledge, a link between metabolically active folate and/or methylcobalamin deficiency and fibromyalgia has not heretofore been established.

Headaches or cephalalgias are defined as pain in the head or upper neck. The over 200 identified types of headaches are generally divisible into two groups. Primary headaches include tension, migraine, and cluster headaches, while secondary headaches are those resulting from an underlying structural problem in the head or neck. Tension headaches are characterized by a dull, pressure-like pain that may radiate from the neck, back, eyes, or other muscle groups in the body and generally last several minutes to a few hours. Migraines tend to be pulsating in character, affect one side of the head, associated with nausea, disabling in severity, and can last from about 3 hours to about 3 days. Tension headaches and migraines are the most common cephalalgias, though their pathophysiology is not well understood. Consequently, these headaches are commonly treated with analgesics. To the best of the inventors' knowledge, a link between metabolically active folate and/or methylcobalamin deficiency and headaches and migraines has not heretofore been established.

HLA-DQ is a cell surface receptor protein comprised of an alpha and beta chain, which are encoded by the HLA-DQA1 and HLA-DQB1 genes, respectively. Certain allelic variants of the DQA1 and DQB1 genes, including DQA1*03, DQA1*05, DQA1*0501, DQA1*0505, DQB1*02, DQB1*0201, DQB1*0202, DQB1*0301, DQB1*0302, DQB1*0303, and DQB1*05, have been associated with a variety of disease states and/or symptoms. Frequently, these alleles are associated with an autoimmune disorder of the small intestine that, in some cases, can manifest as or progress into gluten intolerance or full-blown Celiac Disease. Such autoimmune diseases can occur in genetically predisposed people of all ages. The typical pathogenesis of these diseases begins when a genetically predisposed individual consumes gluten-containing foods. The enzyme tissue transglutaminase modifies the gluten protein such that the immune system cross-reacts with the small-bowel tissue and causes an inflammatory reaction. This can lead to a truncation of the villi lining the small intestine, which interferes with the absorption of nutrients. Symptoms include abdominal cramping, gas and bloating, chronic diarrhea and/or constipation, steatorrhea, vitamin deficiencies, and unexplained weight loss. Additional symptoms can include failure to thrive (in children), depression, anxiety, migraines and/or headaches, blurred vision, and fatigue, miscarriage, peripheral neuropathy, Sjogren's Disease, uveitis, and unexplained ocular pain. The autoimmune dysfunction associated with these genes can also lead to iron deficiency anemia and osteoporosis, and raise the risk of lymphoma. The disorder can manifest at any point in life following triggers such as surgery, viral infection, severe emotional distress, pregnancy and childbirth. The only known effective treatment is a lifelong gluten-free diet. (Di Sabatino, et al., Lancet 373:1480-93 (2009)). To the best of the inventors' knowledge, a link between metabolically active folate and/or methylcobalamin deficiency and these gene alleles has not heretofore been established.

Erectile dysfunction (“ED”) is characterized by the regular or repeated inability to obtain or maintain an erection of the penis. A penile erection is the hydraulic effect of blood entering and being retained in sponge-like bodies within the penis. Though there are no formal tests to diagnose erectile dysfunction, blood tests are generally performed to exclude underlying hormonal diseases such as hypogonadism and prolactinoma. Recognized causes of ED include cardiovascular and peripheral vascular disease, neurological problems, hormonal insufficiencies, and drug side effects. ED may also be a result of poor overall physical health, poor dietary habits, and/or obesity. Diabetes is also considered a risk factor for ED. Additional risk factors for ED include reduced or impaired nitric oxide levels and high levels of homocysteine in the body. To the best of the inventors' knowledge, a link between metabolically active folate and/or methylcobalamin deficiency and erectile dysfunction has not heretofore been established.

Vitamin B deficiencies can have a severe and lasting impact on the physical and mental health of children. Though the clinical manifestations vary widely, early stage symptoms of certain B vitamin deficiencies often include irritability and inability to sleep. (J. Inherit. Metab. Dis. 33:563-70 (2010)). Such deficiencies can progress undetected through early childhood and cause further damage, including developmental delay and impaired vision. (Ronge, et al., Eur. J. Pediatr. 169:241-3 (2010)). If left untreated, the onset of additional symptoms can accelerate and cause irreversible damage such as limb weakness, lack of coordination, idiopathic neuropathy, paresthesiae, and memory and concentration lapses. (Haworth, et al., Am. J. Med. Genet. 45:572-6 (1993)). Moreover, folate and B12 deficiencies have been known to persist even in the presence of normal peripheral folate status. (J. Inherit. Metab. Dis. 33:563-70 (2010); Ronge, et al., Eur. J. Pediatr. 169:241-3 (2010)). To the best of the inventors' knowledge, a link between metabolically active folate and/or methylcobalamin deficiency and idiopathic neuropathy, inability to sleep, inability to concentrate, and/or neurological development issues in children has not heretofore been established.

Regular physical activity, such as strength and endurance training and participation in high-output sports, has a strong positive link with cardiovascular health and induces numerous beneficial adaptations. For instance, increases in blood flow brought on by endurance training can reduce circulating levels of viscosity and haemostatic and inflammatory variables that may interact with increased shear stress, releasing vasoactive substances such as nitric oxide and prostacyclin. (Whyte, et al., Acta Physiol. (Oxf) 199:441-50 (2010)). However, exhausting exercise can cause muscular injuries and inflammation that can jeopardize advances in exercise, fitness, and performance. Specifically, muscle-damaging exercise can increase oxidative stress and damage in blood and skeletal muscle that may persist for several days after exercise. (Nikolidis, et al., Sprots Med., 38:579-606 (2008)). For professional athletes and other highly physically active individuals, the time necessary for muscle recovery from soreness, cramping, and fatigue after training and sports is a crucial factor to continued improvements in fitness and overall success. Consequently, new methods to improve muscle recovery time and/or reduce muscle soreness, cramping, and fatigue are continually sought. To the best of the inventors' knowledge, a link between metabolically active folate and/or methylcobalamin deficiency and muscle recovery and/or repair has not heretofore been established.

Folate is a required nutrient and is frequently added to processed foods, such as cereals and breads, in the form of folic acid. However, folic acid is not itself a generally useful form of folate from a metabolic standpoint. Instead, folic acid is converted, through a series of enzymatic steps, to more metabolically active forms of folate via the folate cycle. In the folate cycle, folic acid is first converted into dihydrofolate (DHF) in the presence of vitamin B3. Also with the aid of vitamin B3, DHF is in turn converted into tetrahydrofolate (THF). THF is then converted into 5,10-methylenetetrahydrofolate (5,10-METH F), either directly or via 5-formiminotetrahydrofolate (5FITHF) and 5,10-methenyltetrahydrofolate intermediates. As a part of this same general process, 5-formyltetrahydrofolate (folinic acid), another folate compound, is also converted into 5,10-METHF, again via a 5,10-methenyltetrahydrofolate intermediate. Finally, 5,10-METHF is converted to 5-methyltetrahydrofolate (5MTHF), also called L-methylfolate, levomefolic acid, levomefolate, and (6S)-5-methyltetrahydrofolate (6S-5MTHF), which is the predominant metabolically active form of folate. (Hasselwander et al., 5-Methyltetrahydrofolate—the active form of folic acid, Functional Foods, 2000 Conference Proceedings, pp 48-59; Kelly et al, Unmetabolized folic acid in serum: acute studies in subjects consuming fortified food and supplements, Am. J. Clin. Nutr., 1997, 65:1790-95.).

While this is the ideal path for metabolism of folic acid, as many as 50% of population may have a reduced ability to effectively convert folic acid into its useable form. (Klerk et al., MTHFR 677 C-T polymorphism and risk of coronary heart disease: A Meta-analysis, JAMA, 2002, 288:2023-30.). Because of this, it is possible to have insufficient amounts of metabolically active folate despite having adequate folic acid intake.

The folate cycle is not isolated, but rather interacts with, and in some cases is intertwined with, other metabolic cycles. For example, the folate cycle interacts with the methylation cycle (also known as the methionine cycle), which produces methionine from homocysteine. More specifically, 5MTHF produced by the folate cycle donates a methyl group, which ultimately allows methionine to be produced from homocysteine. Additionally, the folate cycle interacts with the BH4 cycle, which produces tetrahydrobiopterin (BH4) from dihydrobiopterin (BH2). In this case, the interaction between the cycles involves both cycles utilizing a common enzyme: methylenetetrahydrofolatereductase (MTHFR). Because of these complex interactions, malfunctions in one cycle can cause subsequent malfunctions in the other, related cycles. For example, if an individual has a malfunction in the folate cycle such that insufficient 5MTHF is produced, this can cause a buildup of homocysteine and a deficiency of methionine due to an inability of that individual to use the former to produce the latter.

Vitamin 8-12 is also intimately linked to the folate cycle. For instance, vitamin B-12 is an important cofactor in the metabolism of intermediate folate compounds, as well as being involved in multiple pathways that utilize L-methylfolate. One example of vitamin B-12's involvement in a pathway that involves L-methylfolate is again in the conversion of homocysteine into methionine. As stated above, 5MTHF donates a methyl group that eventually results in conversion of homocysteine into methionine. That methyl group is transferred from 5MTHF to cobalamin, an unmethylated form of vitamin B-12, thereby producing the methyl form of vitamin B-12, methylcobalamin (also called methyl-B12). Methylcobalamin in turn donates the methyl group to homocysteine to convert it into methionine. Thus, if an individual has an inadequate supply of vitamin B-12, the conversion of homocysteine to methionine will be negatively impacted. Vitamin B-12 is also important in other ways, such as being necessary for nerve repair and nerve health. Because of this, deficiencies in vitamin B-12, and methylcobalamin in particular, can lead to serious complications, such as pernicious anemia.

Other vitamin deficiencies are also known to cause a host of malfunctions, pathological conditions, or other difficulties. For instance, vitamin D3 deficiency is known to be related to high blood pressure, diabetes, arthritis, certain autoimmune diseases, and early age-related macular degeneration. (C.D. Meletis, Vitamin D3: Higher Doses Reduce Risk of Common Health Concerns, available at www.vrp.com).

Because the cycles in which many of these nutrients are involved contain multiple enzymatic steps, they are prone to malfunction. Such malfunction can result, for example, from environmental toxins, ingested chemical compounds or toxins, metabolic imbalances, or genetic polymorphisms in the enzymes that carry out the process steps. For instance, the enzyme MTHFR is involved in the folate cycle. More specifically, this enzyme is at least partially responsible for converting 5,10-METHF into 5MTHF. Mutations in the portion of this enzyme that is involved in this conversion are known to exist. One such mutation, the C677T polymorphism, is known to slow down the folate cycle activity of this enzyme, resulting in reduced production of 5MTHF from its precursor product(s). For instance, individuals with this particular polymorphism have reduced CNS L-methylfolate. (Surtees et al., Association of cerebrospinal fluid deficiency of 5-methyltetrahydrofolate, but not S-adenosylmethionine, with reduced concentrations of the acid metabolites of 5-hydroxytryptamine and dopamine, Clinical Science, 1994, 86:697-702). In certain studies, it has been found that approximately 57% of patients with cardiovascular disease have this genetic polymorphism. (Cho et al., The methylenetetrahydrofolate reductase C677T gene mutation is associated with hyperhomocysteinemia, cardiovascular disease and plasma B-type natriuretic peptide levels in Korea, Clin. Chem. Lab. Med., 2006, 44(9):1070-5).

MTHFR is also susceptible to mutation in those portions of the enzyme with activities outside the folate cycle. For instance, another function of MTHFR is the conversion of dihydrobiopterin (BH2) to tetrahydrobiopterin (BH4) in the BH4 cycle. BH4 is subsequently involved in multiple other biological pathways and is essential in the synthesis of numerous catecholamines (e.g., dopamine and noradrenaline/norepinephrine) and indolamines (e.g., serotonin and melatonin), as well nitric oxide synthases, which are involved in immune functions as well as vascularization. As such, a mutation in the portion of MTHFR responsible for BH4 cycle activity, such as the A1298C polymorphism, can cause a disruption in the BH4 pathway and subsequent malfunctions in numerous downstream pathways. For example, the A1298C polymorphism has been associated with glaucoma, with higher incidence of cardiovascular disease, and with subclinical atherosclerosis in rheumatoid arthritis patients. (Shazia et al., MTHFR and A1298C genetic mutation and homocysteine levels in primary open angle and primary closed angle glaucoma, Molecular Vision, 2009, 15:2268-2278; Haviv et al., The common mutations C677T and A1298C in the human methylenetetrahydrofolate reductase gene are associated with hyperhomocysteinemia and cardiovascular disease in hemodialysis patients, Nephron, September 2002, 92(1):120-6; Palo-Morales et al., A1298C polymorphism in the MTHFR gene predisposes to cardiovascular risk in rheumatoid arthritis, Arthritis Res. Ther., 2010, 12:R71).

Moreover, because these multiple cycles are intricately intertwined, a single malfunction can have far-reaching effects. Anything that breaks down the methylation cycle impacts homocysteine levels, nitric oxide levels, affects red blood cell function, increases inflammation, causes immune system malfunctions, causes detoxification system malfunctions, causes antioxidant system malfunctions, and negatively impacts our ability to heal and repair. The results of this are increased lipid oxidation, increased free-radical damage to artery walls, and increased inflammation. All of this has been linked to atherosclerosis and cardiovascular disease.

Because of the fortification of many processed foods, such as cereals and breads, with folic acid, excessive levels of folic acid may exist in much of the human population. For instance, the U.S. National Academy of Sciences recommends a daily intake of 150-600 μg of folic acid depending on the individual's age and pregnancy status. Many folic acid fortified breakfast cereals supply this amount in a single serving, as do many daily multivitamins. In addition, fortified breads frequently supply 5-10% (or more) of the daily requirement in a single slice, while other fortified grains, such as rice, frequently supply 10-20% (or more) of the daily requirement in a single serving. Because of this, it is very common for an individual to have well over twice, and sometimes upwards of four times, the recommended daily intake of folic acid. (USDA National Nutrient Database for Standard Reference, Release 22, Content of Selected Foods per Common Measure, Folate, DFE sorted by nutrient content.).

This is somewhat troubling given that it has been suggested that excessive levels of folic acid might be detrimental in several regards. For instance, some studies have suggested an antagonistic effect of excess folic acid on the metabolically active form by demonstrating an inverse relationship between the amount of unmetabolized folic acid in the blood and the ability of L-methylfolate to cross cell membranes. (Wollack et al., Characterization of folate uptake by choroid plexus epithelial cells in a rat primary culture model, J. Neurochem. 2008; 104:1494-1503; Reynolds, Benefits and risks of folic acid to the nervous system, J. Neurol. Neurosurg. Psychiatry, 2002, 72:567-71.). Further, unmetabolized folic acid has been linked to increased risk of cancer, growth of abnormal cells, increased depression, neurological complications, and decreased immune response. (Troem et al., Unmetabolized Folic Acid in Plasma Is Associated with Reduced Natural Killer Cell Cytotoxicity among Postmenopausal Women, J. Nutr., 2006, 136:189-194; Smith et al., Pteridines and mono-amines: relevance to neurological damage, Postgrad. Med. J., 1986, 62(724):113-23; Asien et al., High-dose B vitamin supplementation and cognitive decline in Alzheimer disease: a randomized controlled trial, JAMA, 2008, 300(15):1774-83.).

To the best of the inventors' knowledge, the presence of unmetabolized folic acid in the body has not heretofore been linked with blood lipid profiles that indicate an increased risk for CAD and/or atherosclerosis. Moreover, while some studies have examined the link between folic acid, homocysteine, and cholesterol (Shidfar, et al., Effect of folate supplementation on serum homocysteine and plasma total antioxidant capacity in hypercholesterolemic adults under Lovastatin treatment: a double-blind randomized controlled clinical trial, Arch. Med. Res., 2009, 40:380-386; VIIIa, et al., L-folic acid supplementation in healthy postmenopausal women: effect on homocysteine and glycolipid metabolism, J. Clin. Endocrinol. Metab., 2005, 90(8):4622-4629; Real, et al., Association of C677T polymorphism in MTHFR gene, high homocysteine and low HDL cholesterol plasma values in heterozygous familial hypercholesterolemia, J. Atheroscler. Thromb., 2009, 16(6):815-820), these studies have not examined the role of metabolically active folate in people with the above genetic polymorphisms. Even further, while it is known that metabolically active folate and active vitamin B-12 may decrease circulating levels of homocysteine, these compounds have not previously been shown to decrease serum cholesterol and triglyceride levels. Additionally, to the best of the inventors' knowledge, the combination of active folate and active vitamin B-12 has not been shown to help prevent or treat fibromyalgia, erectile dysfunction, diseases associated with certain HLA-DQ gene alleles and/or gluten intolerance, or headaches and migraines.

U.S. Patent Application No. 61/358,522, incorporated herein by reference in its entirety, describes the administration of active folate and/or active vitamin-B12 to treat eye disorders in individuals possessing a malfunction in one or more of the folate cycle and BH4 cycle. In the present application, the inventors demonstrate the administration of active folate and, optionally, active vitamin-B12, to treat numerous other disease states in individuals possessing a malfunction in one or more of the folate cycle and BH4 cycle, including abnormal blood lipid profiles. Prevention or treatment of other disease states, such as fibromyalgia; erectile dysfunction; diseases associated with certain HLA-DQ gene alleles and/or gluten intolerance, including Celiac Disease; headaches and/or migraines; muscle recovery and/or repair; as well as idiopathic neuropathy, inability to sleep, inability to concentrate, and/or neurological development issues in children, are also described.

### SUMMARY OF THE INVENTION

In one aspect, the present invention relates to a method of preventing, treating, or otherwise improving one or more disease states related to a metabolic disorder of the folate cycle or BH4 cycle, the method comprising a) identifying a subject organism having one or more metabolic disorders of the folate cycle or BH4 cycle, and b) administering to the subject organism an effective amount of one or more downstream folate compounds and, optionally, methyl-B12. In yet another aspect, the invention further involves administering vitamin B6. In another aspect, the invention further comprises c) decreasing the subject organism's intake of folic acid.

In certain embodiments, the malfunction in one or more of the folate cycle and BH4 cycle is one or more of the C677T and A1298C genetic polymorphisms.

In other embodiments, the one or more disease states are selected from the group consisting of an abnormal blood lipid profile; fibromyalgia; erectile dysfunction; diseases associated with certain HLA-DQ gene alleles and/or gluten intolerance, including Celiac Disease; headaches and/or migraines; muscle soreness, cramping, fatigue, recovery and/or repair in athletes; and idiopathic neuropathy, inability to sleep, inability to concentrate, and/or neurological development issues in children. In particular embodiments, the abnormal blood lipid profile comprises one or more of high total cholesterol, high triglycerides, high LDL, low HDL, or a high LDL to HDL ratio.

In certain other embodiments, the subject organism is a human. In particular embodiments, the subject organism is selected from the group consisting of a child, a diabetic patient, a bariatric patient, a cardiovascular patient, a post surgical patient, and an athlete. In other embodiments, the subject organism is not folic acid deficient.

In further embodiments, the method of administration is selected from the group consisting of an energy drink and a drink designed for diabetics.

In still further embodiments, the one or more downstream folate compounds are selected from the group consisting of DHF, THF, 5FITHF, 5,10-METHF, 5MTHF, and L-methylfolate. In particular embodiments, the one or more downstream folate compounds comprise L-methylfolate. In additional embodiments, the L-methylfolate is provided in a dose of 1 mcg-25 mg/day. In other embodiments, the L-methylfolate is provided in a dose of 1-25 mg/day. In still further embodiments, the methyl-B12 is administered in a dose of 1-2.5 mg/day.

Other features and advantages of the invention will be understood by reference to the drawings, detailed description and examples that follow.

### DETAILED DESCRIPTION OF THE INVENTION

The present invention springs, in part, from the inventor's surprising demonstration that numerous disease states in individuals who possess some type of metabolic malfunction or abnormality associated with folic acid metabolism or intertwined metabolic cycles can be treated by administering to those individuals downstream folate, optionally in combination with one or more of methyl-B12, vitamin B6, and vitamin D3. In U.S. Application No. 61/358,522, entitled “Methods of treating optic disorders,” which is incorporated herein by reference in its entirety, the present inventor demonstrated treatment of optic disorders in such individuals through administration of these compounds. Other disease states or conditions relating to some type of metabolic malfunction or abnormality associated with folic acid metabolism or intertwined metabolic cycles can also be prevented, treated, or otherwise improved through the administration to those individuals of downstream folate, optionally in combination with one or more of methyl-B12, vitamin B6, and vitamin D3, such as abnormal blood lipid profiles; fibromyalgia; erectile dysfunction; diseases associated with certain HLA-DQ gene alleles and/or gluten intolerance, including Celiac Disease; headaches and/or migraines; muscle recovery and/or repair in athletes; as well as idiopathic neuropathy, inability to sleep, inability to concentrate, and/or neurological development issues in children.

In one aspect, the present invention thus relates to methods of preventing, treating, or otherwise improving one or more disease states relating to certain metabolic abnormalities through the administration of downstream folate, optionally in combination with one or more of methyl-B12, vitamin B6, and vitamin D3. In certain embodiments the disease state comprises one or more of an abnormal blood lipid profile; fibromyalgia; erectile dysfunction; diseases associated with certain HLA-DQ gene alleles and/or gluten intolerance, including Celiac Disease; headaches and/or migraines; muscle recovery and/or repair; as well as idiopathic neuropathy, inability to sleep, inability to concentrate, and/or neurological development issues in children. In one particular embodiment, the method comprises administration of L-methylfolate. In other embodiments, the method further involves reducing dietary intake of folic acid. In certain other embodiments, the method further involves administering methyl-B12. In still further embodiments, the method further comprises administering one or more of vitamin B6 and vitamin D3. In still further embodiments, the method involves identifying a subject organism with a malfunction in one or more of the folate or B4 cycles. In certain embodiments, such a malfunction is one or more of the C677T and A1298C genetic polymorphisms. In still further embodiments, the method further involves identifying a subject who is vitamin B12 and D3 deficient and who has elevated levels of homocysteine.

In certain embodiments, the present invention relates to methods of improving blood lipid profiles. In particular embodiments, the invention relates to methods of treating blood lipid profiles that indicate an increased risk for CHD using downstream folate compounds to negate the occurrence of environmental, medication, lifestyle, disease, or genetically induced interference and/or disruption in specific biochemical reactions necessary for maintenance of healthy blood lipid levels. In certain embodiments, methods are provided for improving blood lipid profiles, the methods involving the administration of one or more downstream folate compounds. In one particular embodiment, the method comprises administering L-methylfolate. In other embodiments, the method further involves reducing dietary intake of folic acid. In certain other embodiments, the method further involves administering methyl-B12. In still further embodiments, the method further comprises administering one or more of vitamin B6 and vitamin D3. In still other embodiments, the method further involves first identifying a subject organism with a blood lipid profile that indicates an increased risk for CHD and which individual is not folic acid deficient. In still further embodiments, the method involves identifying a subject organism with a malfunction in one or more of the folate or B4 cycles. In certain embodiments, such a malfunction is one or more of the C677T and A1298C genetic polymorphisms. In still further embodiments, the method further involves identifying a subject who is vitamin B12 and D3 deficient and who has elevated levels of homocysteine.

The entire contents of all references cited in this disclosure are specifically incorporated by reference herein. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

In this disclosure, a number of terms and abbreviations are used. The following definitions are provided.

As used herein, “comprising” is to be interpreted as specifying the presence of the stated features, integers, steps, reagents, or components as referred to, but does not preclude the presence or addition of one or more features, integers, steps or components, or groups thereof. Thus, for example, a composition comprising one downstream folate compound may comprise more downstream folate compounds than those actually recited, i.e., it may comprise two or more distinct downstream folate compounds. Additionally, the term “comprising” is intended to include embodiments encompassed by the terms “consisting essentially of” and “consisting of.” Similarly, the term “consisting essentially of” is intended to include embodiments encompassed by the term “consisting of.”

As used herein, the term “disease state” means any impairment of health or any condition of abnormal function. Disease states related to malfunction of or abnormalities in the folate cycle and/or the BH4 cycle may include eye disorders; abnormal blood lipid profiles; fibromyalgia; erectile dysfunction; diseases associated with certain HLA-DQ gene alleles and/or gluten intolerance, including Celiac Disease; headaches and/or migraines; muscle recovery and/or repair issues in athletes; and idiopathic neuropathy, inability to sleep, inability to concentrate, and/or neurological development issues in children. The term “downstream folate compound” or “downstream folate” means a folate compound downstream of either folic acid or folinic acid in the folate cycle.

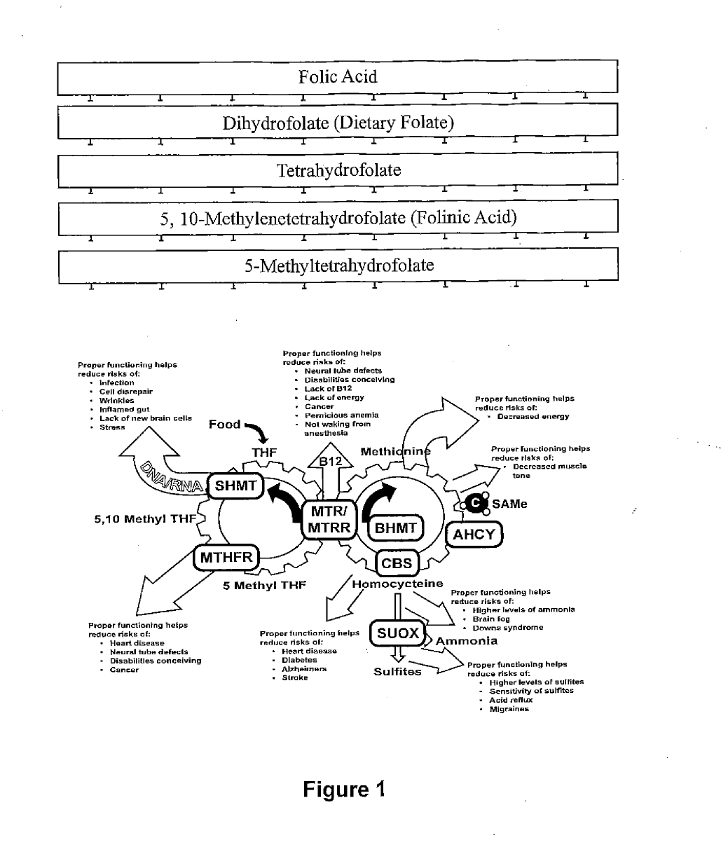

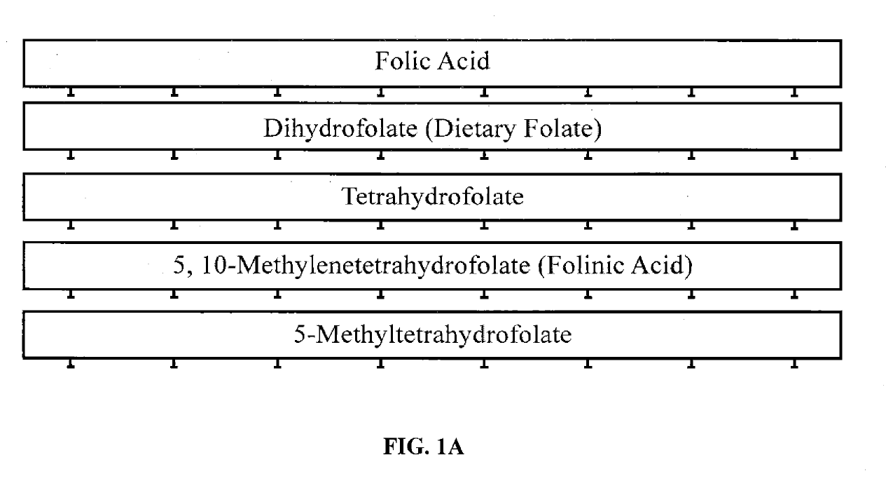

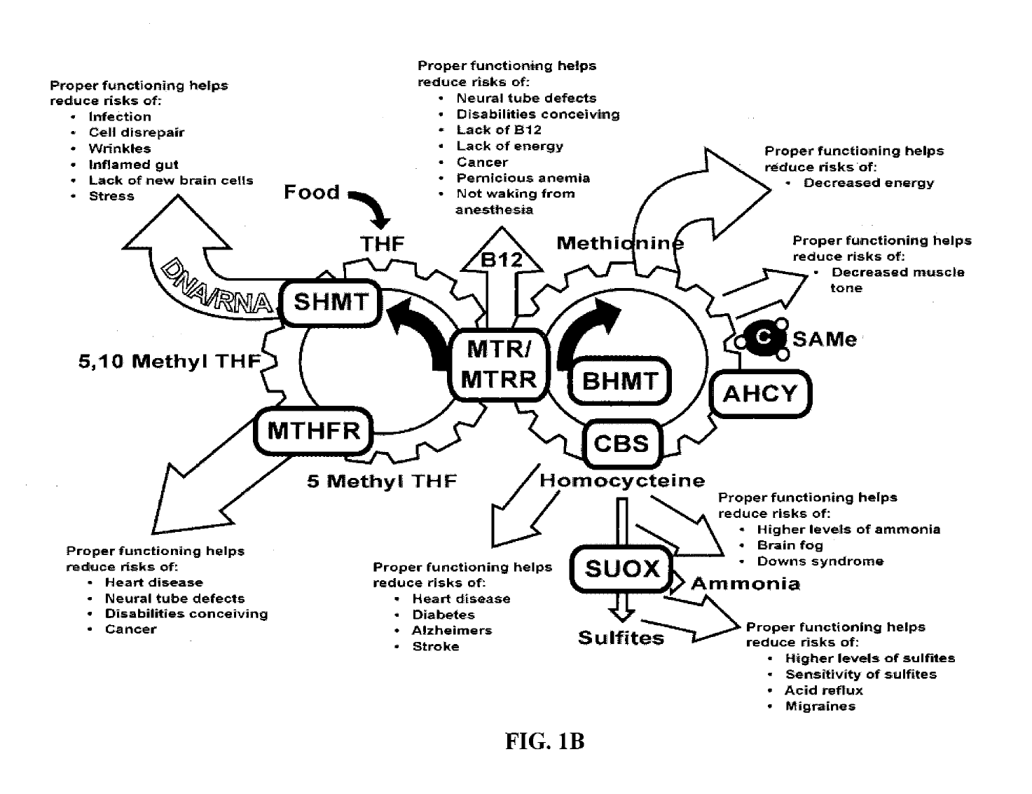

The “folate cycle” refers to the process by which metabolically unrecognizable/inactive folates are converted into metabolically useful/recognizable/active folates in the body. FIG. 1, adapted from a metabolic chart available from KnowYourGenetics.com, shows the major steps and intermediates involved in the folate cycle. As can be seen, during the folate cycle, folic acid is first converted into dihydrofolate (DHF), which is in turn converted to tetrahydrofolate (THF). THF is then converted into 5,10-methylenetetrahydrofolate (5,10-METHF), either directly or via 5-formiminotetrahydrofolate (5FITHF) and 5,10-methenyltetrahydrofolate intermediates. As a part of this same general process, 5-formyltetrahydrofolate (folinic acid), another folate compound, is also converted into 5,10-METHF, again via a 5,10-methenyltetrahydrofolate intermediate. 5,10-METHF is then converted to 5-methyltetrahydrofolate (5MTHF), also called L-methylfolate, levomefolic acid, levomefolate, and (6S)-5-methyltetrahydrofolate (6S-5MTHF), which is the predominant metabolically active form of folate. L-methylfolate is also referred to at various times and/or by various pharmaceutical manufacturers as L-5-Methyltetrahydrfolate, L-5-MTHF, and L-MTHF. The enzyme methylenetetrahydrofolatereductase (MTHFR) is at least partially responsible for converting 5,10-METHF into 5MTHF. Thus, folate compounds downstream of folic acid and folinic acid in the folate cycle include DHF, THF, 5FITHF, 5,10-methenyltetrahydrofolate, 5,10-METHF, 5MTHF, and L-methylfolate. Downstream folate compounds are included, for example, in certain commercially available dietary supplements, including, but not limited to, Metafolin® available from Merck; CerefolinNAC®, Deplin®, and Metanx® available from Pamlab; and Quatrefolic® available from Gnosis.

As used herein, the term “BH4 cycle” means the cycle responsible for the conversion of dihydrobiopterin (BH2) to tetrahydrobiopterin (BH4). One enzyme involved in this cycle is MTHFR.

As used herein, “methyl-B12” refers to methylcobalamin.

As used herein, a “malfunction” in the folate or BH4 cycle means an exogenous or endogenous condition that negatively affects the normal operation of the folate cycle and/or BH4 cycle. Such malfunctions could result, for example, from environmental toxins, ingested chemical compounds or toxins, metabolic imbalances, or genetic disorders, mutations, or polymorphisms affecting enzymes in the folate and/or BH4 cycle, including the C677T and/or A1298C genetic polymorphisms.

As used herein, “C677T” refers to a genetic polymorphism in one or more alleles of a gene encoding the MTHFR enzyme where the cytosine nucleotide at nucleotide position 677 of the MTHFR gene is replaced with a thymine nucleotide. This polymorphism results in a malfunction in the enzyme's folate cycle activity.

As used herein, “A1298C” refers to a genetic polymorphism in one or more alleles of a gene encoding the MTHFR enzyme where the adenine nucleotide at nucleotide position 1298 of the MTHFR gene is replaced with a cytosine nucleotide. This polymorphism results in a malfunction in the enzyme's BH4 cycle activity.

As used herein, “blood lipid profile” means an analysis of the amounts of various types of lipids in the blood. A blood lipid profile can include data for one or more, and preferably all, of a subject organism's total cholesterol level, triglyceride level, LDL level, HDL level, and LDL to HDL ratio.

As used herein, “Coronary Heart Disease” (CHD) refers to the failure of coronary circulation to supply adequate circulation to cardiac muscle and surrounding tissue. As used herein, CHD includes coronary artery disease, which typically results from the accumulation of atheromatous plaques within the walls of the coronary arteries.

A blood lipid profile which indicates an “increased risk for Coronary Heart Disease” is well known to and readily identifiable by persons of ordinary skill in the art. For example, a blood lipid profile which indicates an increased risk for Coronary Heart Disease could include one or more of high total cholesterol, high triglycerides, high LDL, low HDL, or a high LDL to HDL ratio.

As used herein, “improved blood lipid profile” means an improvement in one or more aspects of the blood lipid profile of the subject organism. Such improvements in blood lipid profile are well known to persons of ordinary skill in the art and include, for example, one or more of decreased total cholesterol level, decreased triglyceride level, decreased LDL level, increased HDL level, or decreased LDL to HDL ratio. For instance, an improved blood lipid profile could be a reduction in total cholesterol from a level that is considered high to a level that is considered normal. Similarly, an improved blood lipid profile could be a reduction in serum triglycerides from a level that is considered high to a level that is considered normal.

As used herein, “high cholesterol” means a total serum cholesterol level that is higher than the level that is generally accepted as normal or healthy by persons of ordinary skill in the art. Such levels are generally known to persons of ordinary skill in the art, as are methods for determining a particular subject organism's cholesterol level. In certain embodiments, high cholesterol will include a cholesterol level in the blood of 240 mg/dL or greater. In other embodiments, normal cholesterol will include a cholesterol level in the blood of less than 200 mg/dL.

The term “HDL” refers to high-density lipoproteins that enable lipids like cholesterol and triglycerides to be transported through the blood stream. HDL particles are able to remove cholesterol from atheromas within arteries and transport it back to the liver for excretion or reutilization. As used herein, “low HDL(s)” means a serum HDL level that is lower than the level that is generally accepted as normal or healthy by persons of ordinary skill in the art. Such levels are generally known to persons of ordinary skill in the art, as are methods for determining a particular subject organism's HDL level. In certain embodiments, low HDL(s) refers to an HDL level in the blood below 40 mg/dL (in men) or 50 mg/dL (in women). In other embodiments, normal HDL(s) refers to an HDL level in the blood above 60 mg/dL.

The term “LDL” refers to low-density lipoproteins that enable lipids like cholesterol and triglycerides to be transported through the blood stream. As used herein, “high LDL(s)” means a serum LDL level that is higher than the level that is generally accepted as normal or healthy by persons of ordinary skill in the art. Such levels are generally known to persons of ordinary skill in the art, as are methods for determining a particular subject organism's LDL level. In certain embodiments, high LDL(s) refers to an LDL level in the blood over 160 mg/dL. In other embodiments, normal LDL(s) refers to an LDL level in the blood below 130 mg/dL.

The term “triglyceride” means an ester derived from glycerol and three fatty acids, which is used in the body as an energy source and transporter of dietary fat. As used herein, the term “high triglycerides” means a serum triglyceride level that is higher than the level that is generally accepted as normal or healthy by persons of ordinary skill in the art. Such levels are generally known to persons of ordinary skill in the art, as are methods for determining a particular subject organism's triglyceride level. In certain embodiments, high triglycerides refers to a serum triglyceride level above 200 mg/dL. In other embodiments, normal triglycerides refers to a serum triglyceride level of less than 150 mg/dL.

The term “homocysteine” means an amino acid with the formula HSCH₂CH₂CH(NH₂)CO₂H that is biosynthesized from methionine by the removal of its terminal C^{ε} methyl group. As used herein, the term “high homocysteine” means a serum homocysteine level that is higher than the level that is generally accepted as normal or healthy by persons of ordinary skill in the art. Such levels are generally known to persons of ordinary skill in the art, as are methods for determining a particular subject organism's homocysteine level. In certain embodiments, high homocysteine refers to a serum homocysteine level above 10.4 mg/L (in women) or 11.4 mg/L (in men). In other embodiments, normal homocysteine refers to a serum homocysteine level of less than 8.5 mg/L.

The term “non-folic acid-deficient” means a subject organism that has been found to not have a deficiency in folic acid, i.e., in folic acid as provided in the diet or through dietary supplementation, and as measured in the blood serum following such intake. A determination that a subject organism does not have a folic acid deficiency can be made by any number of suitable tests or analyses to determine folic acid or folate levels in the body. Such tests are well known to persons of ordinary skill in the art and include analysis of folic acid levels in the blood plasma and/or analysis of folic acid levels within red blood cells. In addition, a determination that a subject organism does not have a folic acid deficiency can be made through an analysis of a subject organism's dietary intake of folic acid. If such an analysis reveals that an adequate amount of folic acid is being consumed through the diet, then the subject organism is non-folic acid-deficient. For example, a non-folic acid-deficient subject includes a person who consumes at least the appropriate recommended daily allowance of folic acid as established by the U.S. National Academy of Sciences. In contrast, a folic acid deficient individual would include a person known to consume far below the recommended daily allowance of folic acid.

As used herein, a nutritional “deficiency,” such as a deficiency in vitamin B-12, methyl-B12, vitamin D3, or vitamin B6, means the subject organism possesses a level of the nutrient of interest that is less than the level that is generally accepted as normal by persons of ordinary skill in the art. Such levels are well known to persons of ordinary skill in the art, as are methods for determining a particular subject organism's levels of nutrients of interest. Methods of testing include blood tests for nutrients of interest.

As used herein, “subject organism” means any animal, regardless of species, gender, or age, capable of meeting the other criteria of the invention (e.g., non-folic acid-deficient with high cholesterol). In certain embodiments, the subject organism is a mammal. In certain other embodiments, the subject organism is a companion animal, preferably a dog, cat, horse, or bird. In other embodiments, the subject organism is a human.

As used herein, an “effective amount” is an amount of a compound, such as a downstream folate compound, methyl-B12, vitamin B6, or vitamin D3, that is sufficient to prevent or otherwise cause favorable change a disease state of interest, for example favorable changes in the subject organism's blood lipid profile. For instance, an effective amount of downstream folate includes a sufficient amount of a downstream folate compound to cause a reduction in total serum cholesterol and/or serum triglycerides when provided regularly over a period of days to years.

In certain embodiments, an effective amount of a downstream folate compound is about 1 mcg-25 mg per day, about 1-20 mg per day, about 2.8-15 mg per day, or about 5-10 mg per day. In other embodiments, an effective amount of a downstream folate compound is about 1 mcg per day, about 10 mcg per day, about 20 mcg per day, about 30 mcg per day, about 40 mcg per day, about 50 mcg per day, about 60 mcg per day, about 70 mcg per day, about 80 mcg per day, about 90 mcg per day, about 100 mcg per day, about 200 mcg per day, about 300 mcg per day, about 400 mcg per day, about 500 mcg per day, about 600 mcg per day, about 700 mcg per day, about 800 mcg per day, about 900 mg per day, about 1 mg per day, about 1.5 mg per day, about 2 mg per day, about 2.5 mg per day, about 2.8 mg per day, about 3 mg per day, about 3.5 mg per day, about 4 mg per day, about 4.5 mg per day, about 5 mg per day, about 6 mg per day, about 7 mg per day, about 7.5 mg per day, about 8 mg per day, about 9 mg per day, about 10 mg per day, about 11 mg per day, about 12 mg per day, about 13 mg per day, about 14 mg per day, about 15 mg per day, about 16 mg per day, about 17 mg per day, about 18 mg per day, about 19 mg per day, about 20 mg per day, about 21 mg per day, about 22 mg per day, about 23 mg per day, about 24 mg per day, or about 25 mg per day. In certain other embodiments, an effective amount of a downstream folate compound is about 10 mcg-200 mg per week, about 100 mcg-200 mg per week, about 1-200 mg per week, about 5-150 mg per week, about 10-125 mg per week, about 19.6-105 mg per week, about 20-100 mg per week, about 25-90 mg per week, about 30-80 mg per week, about 35-70 mg per week, about 40-60 mg per week, or about 50-55 mg per week. In other embodiments, an effective amount of a downstream folate compound is about 5-1000 mg per month, about 20-900 mg per month, about 40-800 mg per month, about 50-700 mg per month, about 60-600 mg per month, about 80-500 mg per month, about 100-400 mg per month, about 150-300 mg per month, or about 200-250 mg per month.

In certain embodiments, an effective amount of methyl-B12 is about 1 mcg to about 10 mg per day, about 0.5-5 mg per day, or about 1-2.5 mg per day. In other embodiments, an effective amount of methyl-B12 is about 0.5-100 mg per week, about 3-35 mg per week, or about 7-17.5 mg per week. In other embodiments, an effective amount of methyl-B12 is about 2-300 mg per month, about 15-150 mg per month, or about 30-75 mg per month.

In other embodiments, multiple compounds are administered, such as folate and methyl-B12, or folate, methyl-B12 and one or more of vitamin B6 and D3. In such cases, an effective amount of each component is an amount sufficient to cause favorable changes in the subject organism's blood lipid profile when the components are administered in the desired combination.

It should also be noted that these amounts do not need to be supplied in a single dose, but rather can be supplied in multiple daily, weekly, or monthly doses. For example, dosages can be 1 time per day, 2 times per day, 3 times per day, 4 times per day, 5 times per day, 6 times per day, 7 times per day, 1 time per week, 2 times per week, 3 times per week, 4 times per week, 5 times per week, 6 times per week, 7 times per week, 1 time per month, 2 times per month, 3 times per month, 4 times per month, 5 times per month, 6 times per month, 7 times per month, 8 times per month, 9 times per month, 10 times per month, 11 times per month, 12 times per month, 13 times per month, 14 times per month, 15 times per month, 16 times per month, 17 times per month, 18 times per month, 19 times per month, 20 times per month, 21 times per month, 22 times per month, 23 times per month, 24 times per month, 25 times per month, 26 times per month, 27 times per month, 28 times per month, 29 times per month, 30 times per month, or 31 times per month.

In addition, the administration of the effective amount of one or more downstream folate compounds can continue for a period of days to years. For instance, in certain embodiments, the downstream folate compound(s) are administered on a regular basis for at least 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 2 months, 3 months, 4 months, 5 months, 6 months, 7 months, 8 months, 9 months, 10 months, 11 months, 12 months, 13 months, 14 months, 15 months, 16 months, 17 months 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, or the remaining duration of the subject organism's life. In other embodiments, the downstream folate compound(s) are administered on a regular basis for less than 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 2 months, 3 months, 4 months, 5 months, 6 months, 7 months, 8 months, 9 months, 10 months, 11 months, 12 months, 13 months, 14 months, 15 months, 16 months, 17 months 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, or the remaining duration of the subject organism's life.

The compounds being administered can be supplied in any form and by any route known in the art, for example, orally (e.g., tablet, capsule, liquid, oral suspension, etc.), transdermally (e.g., ointment, patch, etc.), sublingually, subcutaneously, intramuscularly, rectally, in drop form, or intravenously. In certain other embodiments, oral doses can be provided in a time release, delayed release, layered release, or extended release form. In further embodiments, for example where the subject organism is an infant, the compounds can be administered in an infant formula or baby food supplemented with the given compounds. In still further embodiments, for example where the subject organism is a person with diabetes, the compounds can be administered in diabetic drink or food product supplemented with the given compounds. In yet further embodiments, for example where the subject organism is an athlete, the compounds can be administered in a sports or endurance drink, supplement, or food supplemented with the given compounds

Methyl-B12 is available from numerous sources, such as Source Naturals, which supplies methyl-B12, for example, in 5 mg sublingual doses. In certain embodiments, the method of the present invention involves administering 7.5-15 mg L-methylfolate. In still other embodiments, the method of the present invention involves administering 5.6 mg L-methylfolate, 2 mg methylcobalamin (methyl-B12), and 600 mg N-acetylcysteine. In certain other embodiments, the method of the present invention involves administering 3 mg L-methylfolate, 35 mg pyridoxal 5′-phospahte (an active form of vitamin B6), and 2 mg methylcobalamin.

In certain embodiments, the present invention involves improvement of a subject organism's blood lipid profile using one or more downstream folate compounds optionally in combination with methyl-B12 and/or one or more of vitamin B6 and vitamin D3. The present inventor has unexpectedly found that blood lipid profiles that indicate an increased risk for CHD can be effectively improved by administering to those subject organisms an effective amount of one or more downstream folate compounds alone or in combination with one or more of methyl-B12, vitamin B6 and vitamin D3.

To identify subject organisms with a blood lipid profile that indicates an increased risk for CHD, some form of examination and/or testing is typically performed. Such testing is well known to persons of ordinary skill in the art and can include enzymatic blood analysis. In addition, a determination as to whether the subject organism is non-folic acid-deficient can optionally be made through either testing/analysis or review of dietary folic acid intake, as described above.

In other embodiments, the present invention involves treatment of a subject organism suffering from fibromyalgia using one or more downstream folate compounds and optionally one or more of methyl-B12, vitamin B6 and vitamin D3. The present inventor has unexpectedly found that the symptoms of fibromyalgia can be effectively treated by administering to those subject organisms an effective amount of one or more downstream folate compounds alone or in combination with one or more of methyl-B12, vitamin B6 and vitamin D3.

In still further embodiments, the present invention involves treatment of a subject organism suffering from headaches and/or migraines using one or more downstream folate compounds alone or in combination with one or more of methyl-B12, vitamin B6 and vitamin D3. The present inventor has unexpectedly found that headaches and migraines can be effectively treated by administering to those subject organisms an effective amount of one or more downstream folate compounds alone or in combination with one or more of methyl-B12, vitamin B6 and vitamin D3.

In additional embodiments, the present invention relates to treatment of other disorders or disease states associated with or related to a malfunction in the folate and/or BH4 cycle, including, but not limited to, one or more of fibromyalgia; erectile dysfunction; diseases associated with certain HLA-DQ gene alleles and/or gluten intolerance, including Celiac Disease; headaches and/or migraines; muscle recovery and repair issues; and idiopathic neuropathy, inability to sleep, inability to concentrate, and/or neurological development issues in children. Such disorders or disease states can be effectively treated by administering an effective amount of one or more downstream folate compounds alone or in combination with one or more of methyl-B12, vitamin B6 and vitamin D3.

In certain embodiments, testing or other analysis is done to determine whether the subject organism possesses some form of malfunction in the folate and/or BH4 cycles. Such testing is well known to persons of ordinary skill in the art and includes genetic testing to determine the presence or absence of one or both of the C677T and A1298C genetic polymorphisms.

Useful testing to determine whether the subject organism possesses some form of malfunction in the folate and/or BH4 cycles also includes a test to determine if the individual possesses elevated levels of homocysteine, as excess homocysteine can be indicative of a malfunction in the folate cycle due to the interaction between the methionine and folate cycles. Such tests are well known to persons of ordinary skill in the art and include a homocysteine blood test. Another useful test that can be employed is a test to determine whether the individual possesses levels of vitamin B-12 that are within the normal range. In addition to determining whether an individual is vitamin B-12 deficient, such a test is useful, for example, to determine the cause of the elevated homocysteine levels, since elevated homocysteine can also result from a vitamin B-12 deficiency, as vitamin B-12 is also involved in the conversion of homocysteine to methionine. Such tests are well known to persons of ordinary skill in the art and include a vitamin B-12 blood test. In certain preferred embodiments, a homocysteine blood test will be performed along with a vitamin B-12 blood test and an analysis of the individual's dietary folate intake to determine whether the individual 1) consumes adequate folic acid, and therefore is not folic acid deficient, 2) has vitamin B-12 levels that are within the normal range, and therefore is not vitamin B-12 deficient, and 3) possesses excess homocysteine, and therefore, in light of the results of 1) and 2), appears to have a malfunction in the folate pathway. Testing can also be done to determine if the individual is vitamin D deficient, such as a 25-hydroxy vitamin D blood test.

Treatment of a subject organism according to the present invention is accomplished by supplying that subject organism with an effective amount of one or more downstream folate compounds. As described above, such compounds can include one or more of DHF, THF, 5FITHF, 5,10-METHF, 5MTHF, and L-methylfolate. Also as discussed above, the downstream folate compound(s) can be administered in one or more doses at regular intervals for a period of days to years.

In addition to administering downstream folate compound(s), in certain embodiments, the method further involves reducing the subject organism's folic acid intake. Such a reduction in intake can be accomplished in any suitable manner. Methods for reducing folic acid intake are well known to persons of ordinary skill in the art and include reducing the amount of folic acid consumed through dietary supplements and/or reducing the intake of folic acid fortified foods, such as processed foods, including fortified breads and cereals.

Thus, in one embodiment, the method involves identifying a subject organism having a disorder or disease state associated with or related to a malfunction of one or more of the folate or BH4 cycles, and administering to that subject organism, an effective amount of L-methylfolate. In certain embodiments, the amount of L-methylfolate administered is 1 mcg to 25 mg per day. In other embodiments, the method further comprises decreasing the subject organism's intake of folic acid. In certain embodiments, folic acid intake is decreased by 1-4 mg per day. In further embodiments, the method involves administering an effective amount of methyl-B12 in conjunction with folate. In certain embodiments, the methyl-B12 is administered in an amount of 1-2.5 mg per day. In still further embodiments, folate and methyl-B12 are administered in conjunction with one or more of vitamin B6 and vitamin D3. In yet further embodiments, the method further involves determining whether the subject individual, or one or both parents of the subject organism, possesses one or both of the C677T and A1298C genetic polymorphisms. In certain embodiments, the disorder or disease state is selected from the group consisting of abnormal blood lipid profile; fibromyalgia; erectile dysfunction; diseases associated with certain HLA-DQ gene alleles and/or gluten intolerance, including Celiac Disease; headaches and/or migraines; muscle recovery and repair issues; and idiopathic neuropathy, inability to sleep, inability to concentrate, and/or neurological development issues in children.

In one particular embodiment, the method involves identifying a person having a blood lipid profile that indicates an increased risk for CHD, and administering to that person 1 mcg to 25 mg per day of L-methylfolate. In other embodiments, the method further involves determining whether the person possesses one or both of the C677T and A1298C genetic polymorphisms. In other embodiments, the method further involves decreasing the person's intake of folic acid, for example by 1-4 mg per day. In certain other embodiments, the method further involves administering an effective amount of methyl-B12, for example 1-2.5 mg per day. In certain other embodiments, the method involves 1) identifying a subject organism with a blood lipid profile that indicates an increased risk for CHD; 2) testing the subject organism to determine if it possesses one or both of the C677T and A1298C polymorphisms; 3) testing the subject organism to determine if it possesses above normal homocysteine levels and below normal vitamin B12 and vitamin D levels; and 4) administering to the subject organism an effective amount of a downstream folate compound and methyl-B12 and, optionally, one or more of vitamin B6 and vitamin D3.

In another particular embodiment, the method involves identifying an individual who possess one or more of the HLA-DQ gene alleles DQA1*03, DQA1*05, DQA1*0501, DQA1*0505, DQB1*02, DQB1*0201, DQB1*0202, DQB1*0301, DQB1*0302, DQB1*0303, and DQB1*05, or an individual demonstrating symptoms of a disease state related to those alleles, and administering to that person 1 mcg to 25 mg per day of L-methylfolate. In other embodiments, the method further involves determining whether the person possesses one or both of the C677T and A1298C genetic polymorphisms. In other embodiments, the method further involves decreasing the person's intake of folic acid, for example by 1-4 mg per day. In certain other embodiments, the method further involves administering an effective amount of methyl-B12, for example 1-2.5 mg per day. In certain other embodiments, the method involves 1) identifying a subject organism who possess one or more of the HLA-DQ gene alleles DQA1*03, DQA1*05, DQA1*0501, DQA1*0505, DQB1*02, DQB1*0201, DQB1*0202, DQB1*0301, DQB1*0302, DQB1*0303, and DQB1*05, and/or a subject organism demonstrating symptoms of a disease state related to those alleles; 2) testing the subject organism to determine if it possesses one or both of the C677T and A1298C polymorphisms; 3) testing the subject organism to determine if it possesses above normal homocysteine levels and below normal vitamin B12 and vitamin D levels; and 4) administering to the subject organism an effective amount of a downstream folate compound and methyl-B12 and, optionally, one or more of vitamin B6 and vitamin D3.

In yet another particular embodiment, the method involves identifying a person demonstrating symptoms of fibromyalgia, and administering to that person 1 mcg to 25 mg per day of L-methylfolate. In other embodiments, the method further involves determining whether the person possesses one or both of the C677T and A1298C genetic polymorphisms. In other embodiments, the method further involves decreasing the person's intake of folic acid, for example by 1-4 mg per day. In certain other embodiments, the method further involves administering an effective amount of methyl-B12, for example 1-2.5 mg per day. In certain other embodiments, the method involves 1) identifying a subject organism with symptoms of fibromyalgia; 2) testing the subject organism to determine if it possesses one or both of the C677T and A1298C polymorphisms; 3) testing the subject organism to determine if it possesses above normal homocysteine levels and below normal vitamin B12 and vitamin D levels; and 4) administering to the subject organism an effective amount of a downstream folate compound and methyl-B12 and, optionally, one or more of vitamin B6 and vitamin D3.

In still another particular embodiment, the method involves identifying a person demonstrating chronic or recurring headaches or migraines, and administering to that person 1 mcg to 25 mg per day of L-methylfolate. In other embodiments, the method further involves determining whether the person possesses one or both of the C677T and A1298C genetic polymorphisms. In other embodiments, the method further involves decreasing the person's intake of folic acid, for example by 1-4 mg per day. In certain other embodiments, the method further involves administering an effective amount of methyl-B12, for example 1-2.5 mg per day. In certain other embodiments, the method involves 1) identifying a subject organism with chronic or recurring headaches or migraines; 2) testing the subject organism to determine if it possesses one or both of the C677T and A1298C polymorphisms; 3) testing the subject organism to determine if it possesses above normal homocysteine levels and below normal vitamin B12 and vitamin D levels; and 4) administering to the subject organism an effective amount of a downstream folate compound and methyl-B12 and, optionally, one or more of vitamin B6 and vitamin D3.

Following treatment, the effectiveness of the treatment can be determined by again administering some form of testing or examination to determine the existence or severity of the disease state. For instance, following treatment, the subject's blood lipid profile can be examined, wherein a reduction in one or more of total cholesterol level, triglyceride level, LDL level, or LDL to HDL ratio, or an increase in HDL level, indicates that the treatment method was effective. Such an improvement in a blood lipid profile can be readily determined by persons of ordinary skill in the art.

### EXAMPLES

The present invention is further defined in the following Examples. It should be understood that these Examples, while indicating preferred embodiments of the invention, are given by way of illustration only. The present invention is not limited to the embodiments described and exemplified below, but is capable of variation and modification within the scope of the appended claims.

### Example 1

Patient 1 was a 61-year-old female. It was determined through blood testing that her triglyceride, total cholesterol, and HDL levels were 542, 207, and 44 mg/dL, respectively. A detailed dietary history indicated that she was not at risk for folic acid deficiency. Through genetic testing, it was determined that the patient had one or both of the C677T and A1298C polymorphisms. In addition, tests were performed to determine the levels of folate, vitamin B12, and homocysteine in the body. The patient was identified as having low folate and B12 levels, and high homocysteine levels. The patient was placed on a regimen of 7.5 mg L-methylfolate and 1000 mcg methyl-B12 per day, and told to reduce intake of processed foods as much as possible, such as by switching to organic foods. After one month, she was reexamined and found to have triglyceride, total cholesterol, and HDL levels of 177, 143, and 46.8 mg/dL, respectively. The patient was then placed on a regimen of 7.5 mg L and D-methylfolate and 1000 mcg methyl-B12 per day for 30 days. The patient was reexamined and found to have triglyceride, total cholesterol, and HDL levels of 591, 204, and 33 mg/dL, respectively.

### Example 2

Patient 2 was a 62-year-old female. It was determined through blood testing that her triglyceride, total cholesterol, and HDL levels were 221, 160, and 37 mg/dL, respectively. A detailed dietary history indicated that she was not at risk for folic acid deficiency. Through genetic testing, it was determined that the patient had one or both of the C677T and A1298C polymorphisms. In addition, tests were performed to determine the levels of folate, vitamin B12, and homocysteine in the body. The patient was identified as having low folate and B12 levels, and high homocysteine levels. The patient was placed on a regimen of 7.5 mg L-methylfolate and 1000 mcg methyl-B12 per day, and told to reduce intake of processed foods as much as possible, such as by switching to organic foods. After eight weeks, she was reexamined and found to have triglyceride, total cholesterol, and HDL levels of 133, 139, and 43.7 mg/dL, respectively. The patient was then placed on a regimen of 7.5 mg L and D-methylfolate and 1000 mcg methyl-B12 per day for 30 days. The patient was reexamined and found to have triglyceride, total cholesterol, and HDL levels of 155, 142, and 25 mg/dL, respectively.

### Example 3

Patient 3 was a 54-year-old male taking cholesterol medication. It was determined through blood testing that his triglyceride, total cholesterol, and HDL levels were 204, 185, and 49 mg/dL, respectively. A detailed dietary history indicated that he was not at risk for folic acid deficiency. Through genetic testing, it was determined that the patient had one or both of the C677T and A1298C polymorphisms. In addition, tests were performed to determine the levels of folate, vitamin B12, and homocysteine in the body. The patient was identified as having low folate and B12 levels, and high homocysteine levels. The patient was placed on a regimen of 7.5 mg L-methylfolate and 1000 mcg methyl-B12 per day, and told to reduce intake of processed foods as much as possible, such as by switching to organic foods. The patient was reexamined after approximately three months and found to have triglyceride, total cholesterol, and HDL levels of 119, 192, and 64 mg/dL, respectively. The patient was again reexamined after another approximately three months and found to have triglyceride, total cholesterol, and HDL levels of 147, 197, and 64 mg/dL, respectively.

### Example 4

Patient 4 was a 57-year-old male. It was determined through blood testing that his triglyceride, total cholesterol, and HDL levels were 198, 231, and 38 mg/dL, respectively. A detailed dietary history indicated that he was not at risk for folic acid deficiency. Through genetic testing, it was determined that the patient had one or both of the C677T and A1298C polymorphisms. In addition, tests were performed to determine the levels of folate, vitamin B12, and homocysteine in the body. The patient was identified as having low folate and B12 levels, and high homocysteine levels The patient was placed on a regimen of 7.5 mg L-methylfolate and 1000 mcg methyl-B12 per day, and told to reduce intake of processed foods as much as possible, such as by switching to organic foods. After one month, he was reexamined and found to have triglyceride, total cholesterol, and HDL levels of 157, 162, and 48 mg/dL, respectively. The patient was then reexamined 18 months later and found to have triglyceride, total cholesterol, and HDL levels of 165, 133, and 48 mg/dL, respectively.

### Example 5

Patient 5 was a 49-year-old female. It was determined through blood testing that her triglyceride, total cholesterol, and HDL levels were 431, 211, 37 mg/dL, respectively. A detailed dietary history indicated that she was not at risk for folic acid deficiency. Through genetic testing, it was determined that the patient had one or both of the C677T and A1298C polymorphisms. In addition, tests were performed to determine the levels of folate, vitamin B12, and homocysteine in the body. The patient was identified as having low folate and B12 levels, and high homocysteine levels. The patient was placed on a regimen of 7.5 mg L-methylfolate and 1000 mcg methyl-B12 per day, and told to reduce intake of processed foods as much as possible, such as by switching to organic foods. After one year, she was reexamined and found to have triglyceride and total cholesterol levels of 650 and 328 mgldL, respectively, and an HDL level that was too low to detect. An oral interview revealed that the patient had failed to comply with the prescribed treatment regimen. The patient was advised of the importance of daily compliance and subsequently agreed to strictly comply with the prescribed regimen of 7.5 mg L-methylfolate and 1000 mcg methyl-B12 per day. She was reexamined two months later and found to have triglyceride, total cholesterol, and HDL levels of 572, 241, and 31 mg/dL, respectively. The patient was again reexamined three months later and found to have triglyceride, total cholesterol, and HDL levels of 51, 143, and 58 mg/dL, respectively.

### Example 6

Five patients of mixed age, sex (F=female; M=male) and race (B Black; W=White, Non-Hispanic; H=Hispanic) presented with abnormal blood lipid profiles (Baseline Labs; C=Total cholesterol; Tri=Total Triglycerides) and a history of hypercholesterolemia (see Table 1). A detailed dietary history indicated that the patients were not at risk for folic acid deficiency. Through genetic testing, it was determined that the patients had one or both of the C677T and A1298C polymorphisms. The patients were placed on a twice per day regimen of L-methylfolate, methyl-B12, and pyridoxal-5-phosphate (P5P; active vitamin B6), as well as a once per day dose of Vitamin D3. Follow-up lab work was performed which demonstrated an improved blood lipid profile, as shown in Table 1.

TABLE 1
Treatment of patients presenting with abnormal blood lipid profiles

| | | | | | History of | | | | | |
|---|---|---|---|---|---|---|---|---|---|---|
| Patient | | | | | Hypercho- | | | Baseline | Follow-up | |
| No. | Age | Weight | Sex | Race | lesterolemia | Medications | MTHFR | Labs | Labs | Improvement |
| 27769 | 65 | 170.2 | F | B | 10 years | Hyzar, | A1298C | Oct. 28, 2010 | Mar. 7, 2011 | Decrease in |
| | | | | | | Lopressor, | | C—243, | C—183, | Total |
| | | | | | | Maxide, Vit | | Tri—65, | Tri—65, | Cholesterol, |
| | | | | | | D, Vytorin | | HDL—89, | HDL—104, | Increase in |
| | | | | | | | | LDL—93 | LDL—123 | HDL, |
| | | | | | | | | | | Decrease in |
| | | | | | | | | | | LDL. |
| 14660 | 54 | 200 | M | W | 3 years | PPI, Celexa | C677T | Oct. 18, 2010 | Jun. 15, 2011- | Decrease in |
| | | | | | | | | C—200, | C—190, | Total |
| | | | | | | | | Tri—95, | Tri—62, | Cholesterol, |
| | | | | | | | | HDL—47, | HDL—57, | Increase in |
| | | | | | | | | LDL—134 | LDL—120 | HDL, |
| | | | | | | | | | | Decrease in |
| | | | | | | | | | | LDL and |
| | | | | | | | | | | Triglycerides. |
| 42405 | 49 | 220 | M | W | 3 years | Suboxone, | C677T | Nov. 4, 2010 | Mar. 24, 2011 | Decrease in |
| | | | | | | Valium, | | C—265, | C—193, | Total |
| | | | | | | Metforminl | | Tri—308, | Tri—129, | Cholesterol, |
| | | | | | | | | HDL—39, | HDL—73, | Increase in |
| | | | | | | | | LDL—164 | LDL—98 | HDL, |
| | | | | | | | | | | Decrease in |
| | | | | | | | | | | LDL and |
| | | | | | | | | | | Triglycerides. |
| 26847 | 50 | 176 | M | H | 2 years | Crestor, | C677T | Jun. 7, 2011 | Aug. 24, 2011 | Came off |
| | | | | | | ASA, Fish | | C—233, | C—172, | Crestor at |
| | | | | | | Oil | | Tri—656, | Tri—217, | Baseline |
| | | | | | | | | HDL—38, | HDL—48, | Labs, |
| | | | | | | | | LDL—Too High | LDL—81 | Decrease in |
| | | | | | | | | to Count, | | Total |
| | | | | | | | | Greater | | Cholesterol, |
| | | | | | | | | than 400 | | Increase in |
| | | | | | | | | | | HDL, |
| | | | | | | | | | | Decrease in |
| | | | | | | | | | | Triglycerides |
| | | | | | | | | | | and LDL. |
| 17560 | 59 | 150.6 | F | W | 17 years- | Lorcet Plus, | A129SC | Sep. 13, 2010 | Mar. 7, 2011 | Decrease in |
| | | | | | cannot | zanaflex, | | C—225, | C—200, | Total |
| | | | | | tolerate | ambien | | Tri—189, | Tri—72, | Cholesterol, |
| | | | | | statins | | | HDL—61, | HDL—104, | Increase in |
| | | | | | | | | LDL—126 | LDL—123 | HDL, |
| | | | | | | | | | | Decrease in |
| | | | | | | | | | | LDL and |
| | | | | | | | | | | Triglycerides. |

### Example 7

Patient 7 was a 62-year-old female that presented with classic fibromyalgia symptoms. A detailed dietary history indicated that she was not at risk for folic acid deficiency. Through genetic testing, it was determined that the patient had one or both of the C677T and A1298C polymorphisms. In addition, tests were performed to determine the levels of folate and vitamin B12 in the body. The patient was identified as having low folate and B12 levels. The patient was placed on a regimen of 5 mg L-methylfolate and 2 mg methyl-B12 per day. She was reexamined four months later and reported improvement in fibromyalgia symptoms.

### Example 8

Patient 8 was a 64-year-old female that presented with classic fibromyalgia symptoms. A detailed dietary history indicated that she was not at risk for folic acid deficiency. Through genetic testing, it was determined that the patient had one or both of the C677T and A1298C polymorphisms. In addition, tests were performed to determine the levels of folate and vitamin B12 in the body. The patient was identified as having borderline low folate and B12 levels. The patient was placed on a regimen of 3 mg L-methylfolate per day. She was reexamined one-month later and reported improvement in fibromyalgia symptoms.

### Example 9

Patient 9 was a 57-year-old female that presented with classic fibromyalgia symptoms. A detailed dietary history indicated that she was not at risk for folic acid deficiency. Through genetic testing, it was determined that the patient had one or both of the C677T and A1298C polymorphisms. The patient was placed on a regimen of 7.5 mg L-methylfolate and 2 mg methyl-B12 per day. She was reexamined two months later and reported improvement in fibromyalgia symptoms.

### Example 10

Patient 10 was a 75-year-old female that presented with classic symptoms of gluten intolerance, including blurred vision, neuropathy, constipation, and anxiety. Through genetic testing, it was determined that the patient had one or both of the C677T and A1298C polymorphisms. In addition, tests were performed to determine the levels of folate and vitamin B12 in the body. The patient was identified as having low folate and B12 levels. The patient was placed on a gluten-free diet and a regimen of 5 mg L-methylfolate and 2 mg methyl-B12 per day. She was reexamined two months later and reported improvements in numbness, anxiety, and constipation.

### Example 11

Patient 11 was a 59-year-old female that presented with classic symptoms of gluten intolerance, including blurred vision, chronic pain, eye pain, weakness, lack of energy, and poor nutrient absorption. Through genetic testing, it was determined that the patient had one or both of the C677T and A1298C polymorphisms. In addition, tests were performed to determine the levels of folate and vitamin B12 in the body. The patient was identified as having low folate and B12 levels. The patient was placed on a gluten-free diet and a regimen of 5 mg L-methylfolate and 2 mg methyl-B12 per day. She was reexamined six weeks later and reported improvement in most symptoms.

### Example 12

Patient 12 was a 54-year-old female that presented with classic symptoms of gluten intolerance, including neuropathy and skin sores. Through genetic testing, it was determined that the patient had one or both of the C677T and A1298C polymorphisms. In addition, tests were performed to determine the levels of folate and vitamin B12 in the body. The patient was placed on a gluten-free diet and a regimen of 5 mg L-methylfolate and 2 mg methyl-B12 per day. She was reexamined three months later and reported improvement in all symptoms.

### Example 13

Patient 13 is an 82-year-old white female with a history of cortical occipital stroke, Lupus, chronic irritable bowel symptoms, and severe Sjogren's Syndrome with extreme superficial punctate keratitis causing vision loss down to 20/200 and 20/400 with severe discomfort. All conventional therapies including steroids and lubricants failed over a 4-year period. A corneal melt began and reached 90% loss of corneal thickness. Through genetic testing, it was determined that the patient had both of the C677T and A1298C polymorphisms. Genetic testing for HLA status revealed that the patient was HLA A1*05/B1*02. A daily regimen of L-methylfolate, methyl-B12, and Vitamin D was begun and a gluten free diet prescribed. The patient's corneal melt improved and the severe punctate keratitis resolved, leaving the patient with vision of 20/30 and 20/50.

Following this improvement, the patient's pharmacy switched the medication being administered from the original purified L-methylfolate product to one containing 8% R-methylfolate impurities. Shortly thereafter, the patient presented with blurred vision, hypertension, and congestive heart failure, resulting in a prolonged admission to a hospital intensive care unit. While in the hospital, the patient's medication was again returned to the original purified L-methylfolate product, resulting in recovery from all symptoms. Upon being released from the hospital, the patient again resumed taking the medication that contained 8% R-methylfolate impurities, which resulted in a recurrence of the hypertension and congestive heart failure and a second hospitalization. Once again, the hospital administered a purified L-methylfolate product, resulting in recovery from the symptoms. Upon release from the hospital, the patient continued taking the originally prescribed L-methylfolate product, which resulted in a stabilization of her cardiovascular system and a return of her vision and ocular surface disease to their improved state.

### Example 14

Patient 14 is a 49-year old white female with a history of severe eye pain and dryness of unknown etiology. The patient had a history of traumatic cataract surgery with endophthalmitis and loss of the eye. She now has a well-fitted prosthesis. Attempted treatments for the eye pain and dryness have included topical lubrication, tear duct plugs, and a variety of topical treatments, all with no success. The patient also presented with high blood pressure and chronic severe diarrhea that had been ongoing for numerous years and that had defied medical diagnosis. Genetic testing revealed that the patient was homozygous for the A1298C polymorphism. Genetic testing for HLA status revealed that the patient was HLA A1*05/B1*02. A gluten free diet was prescribed along with daily a daily regimen of L-methylfolate and methyl-B12. Over a one-month period, the patient's severe eye pain and chronic diarrhea resolved and for three years she has remained symptom free as long as the diet is adhered to and L-methylfolate/methyl B12 regimen is continued.

### Example 15

Patient 15 is a 60-year-old white female with a multiyear history of dry eye discomfort, chronic diarrhea, chronic weakness, memory loss, chronic pain, severe migraine headaches requiring heavy pain medications interfering with driving and work, and numbness of her extremities. Genetic testing revealed that the patient was homozygous for the C677T polymorphism. Genetic testing for HLA status revealed that the patient was HLA A1*05/B1*02. The patient was prescribed a gluten free diet and placed on a daily regimen of purified L-methylfolate and methyl-B12. The patient showed immediate improvement to her eye dryness and discomfort, migraine headaches, peripheral numbness, and chronic diarrhea, which improvement continued for approximately one year. Approximately one year from the beginning of treatment, the patient's pharmacy made an unauthorized switch from the purified L-methylfolate product to one containing 8% R-methylfolate impurities. This change in medication resulted in a return of the severe migraine headaches, as well as the patient reporting being in a mental fog. After six weeks on the impure methylfolate product, the medication change was discovered and the administration of that product was discontinued, which resulted in a partial resolution of the headaches. When the administration of the purified L-methylfolate product was resumed, the migraine headache and mental fog symptoms were resolved.

### Example 16

Patient 16 is a 53-year-old white female who presented with acute inset blurred vision in one eye, malignant hypertension, vitreous hemorrhage, bilateral small branch vein occlusions, and localized retinal neovascularization requiring emergency treatment systemically, and a quadrantal PRP was required. The patient was placed on a daily regimen of L-methylfolate and methyl-B12. A more in-depth medical history revealed numbness of the hands and irritable bowel symptoms with chronic constipation. Genetic testing revealed that the patient was homozygous for the C677T polymorphism. Genetic testing for HLA status revealed that the patient was HLA A1*05/B1*02. The patient was prescribed a gluten free diet and daily administration of L-methylfolate and methyl-B12 were continued. Following continued use of these treatments, the patient's Retinopathy, hypertension, and bowel issues resolved or improved.

### Example 17

Patient 17 is a 17-year old white female who presented with a one-year history of painful foreign body sensation in her eyes, chronic diarrhea, memory fog, difficulty in school, burning and tingling in the legs, and rashes and skin ulcerations over her body. The patient's mother was known to have Celiac Disease. A Schirmer's test was administered, with results of 18 and 26 in each of her eyes. Genetic testing revealed that the patient was homozygous for the A1298C polymorphism. Genetic testing for HLA status revealed that the patient was HLA A1*05/B1*02. The patient was prescribed a gluten free diet and a daily regimen of L-methylfolate and methyl-B12. This treatment resulted in improvement of ocular symptoms, rash, paresthesias, mental fog, and schoolwork.

### Example 18

Patient 18 is a 42-year-old white female who presented with a several year history of ocular irritation and narrow angles requiring laser peripheral iridectomy. Post-laser treatment, her eye pain became worse and chronic, accompanied with episcleritis, and superficial punctate keratitis. The patient reports a family history of thyroid disease, lupus, and diabetes, and further reports that her son bloats when he eats pasta. Lab testing revealed that the patient had a Vitamin D deficiency (10.0 ng/mL). Genetic testing revealed that the patient was heterozygous for both the A1298C and C677T polymorphisms. Genetic testing for HLA status revealed that the patient was HLA DQA1*01/DQ B1*0501, a rare known genotype for Celiac Disease. A daily regimen of L-methylfolate, methyl-B12, and Vitamin D were begun and a gluten free diet was prescribed. This treatment resulted in improvement of the patient's episcleritis and superficial punctate keratitis as long as the patient stayed on the prescribed diet and daily regimen.

### Example 19

Eleven patients presented with chronic headaches and/or migraines. It was determined through genetic testing that each patient had one or both of the C677T and A1298C polymorphisms. Each patient was placed on a once or twice daily regimen of L-methylfolate (see Table 2) plus 2 mg methyl-B12 and 25 mg vitamin B6. All patients were evaluated 30 days, 3 months, 6 months and 1 year after beginning the treatment regimen or until symptoms resolved. All patients demonstrated improvement with each subsequent evaluation. Over 90% of the patients realized complete cessation of headaches and migraines within six months of regularly taking L-methylfolate, methyl-B12, and vitamin B6.

TABLE 2
Dosage regimens and recovery times for patients
presenting with chronic headaches and migraines.

| | | | Time until | |
|---|---|---|---|---|
| Age, Gender | Start Date | Dosage | Recovery | |
| 32, female | Jul. 10, 2009 | 2.8 g twice a day | 30 | days |
| 16, female | Apr. 27, 2009 | 2.8 g twice a day | 60 | days |
| 8, male | Aug. 03, 2009 | 2.8 g twice a day | 30 | days |
| 19, male | Feb. 19, 2009 | 2.8 g twice a day | 6 | months |
| 34, female | May 19, 2009 | 2.8 g twice a day | 30 | days |
| 11, female | Dec. 29, 2008 | 7.5 g once a day | 30 | days |
| 9, female | Mar. 30, 2009 | 2.8 g twice a day | 6 | months |
| 51, female | Dec. 19, 2008 | 2.8 g twice a day | 1 | year |
| 20, female | Sep. 05, 2008 | 2.8 g twice a day | 6 | months |
| 31, female | Mar. 19, 2009 | 2.8 g twice a day | 3 | months |
| 60, male | Feb. 20, 2009 | 2.8 g twice a day | 3 | months |

### Example 20

Patient 20 was a 58-year-old male that presented with erectile dysfunction. Erectile dysfunction medication (Viagra® 50 mg for 2 months) had been ineffective. Through genetic testing, it was determined that the patient had one or both of the C677T and A1298C polymorphisms. The patient was placed on a regimen of 5,0 mg L-methylfolate and 2 mg methyl-B12 per day. He was reexamined two months later and reported that his symptoms had resolved.

### Example 21

Patient 21 was a 30-year-old male that presented with erectile dysfunction. Erectile dysfunction medication (Viagra® 20 mg for 1 month) had been ineffective. Through genetic testing, it was determined that the patient had one or both of the C677T and A1298C polymorphisms. The patient was placed on a regimen of 2.8 mg L-methylfolate and 2 mg methyl-B12 per day. He was reexamined 30 days later and reported improvement of symptoms.

### Example 22

Patient 22 was a 57-year-old male that presented with erectile dysfunction. Through genetic testing, it was determined that the patient had one or both of the C677T and A1298C polymorphisms. The patient was placed on a regimen of 5.0 mg L-methylfolate and 2 mg methyl-B12 per day. He was reexamined 30 days later and reported improvement of symptoms.

### Example 23

Patient 23 was a 62-year-old white male that presented with erectile dysfunction. Past medical history also indicated hyperlipidemia and neuropathy. Patient had been taking sildenafil citrate and tadalafil with no resolution of the erectile dysfunction. Through genetic testing, it was determined that the patient had both of the C677T and A1298C polymorphisms. The patient was placed on a regimen of L-methylfolate, methyl-B12, and pyridoxal-5-phosphate (PSP; active vitamin B6) twice per day. After two months of treatment, the patient reports resolution of all symptoms—no erectile dysfunction issues.

### Example 24

Patient 24 was a 51-year-old Hispanic male that presented with erectile dysfunction. Past medical history also indicated hyperlipidemia and prostate cancer. Patient had been taking sildenafil citrate and tadalafil with no resolution of the erectile dysfunction. Through genetic testing, it was determined that the patient had one or both of the C677T and A1298C polymorphisms. The patient was placed on a regimen of L-methylfolate, methyl-B12, and pyridoxal-5-phosphate (P5P; active vitamin B6) twice per day. After one month of treatment, the patient reports resolution of all symptoms—no erectile dysfunction issues.

### Example 25

Patient 25 was a 6-year-old boy with a history of idiopathic neuropathy, inability to sleep, inability to concentrate, fatigue, and reduced and blurry vision. Through genetic testing, it was determined that the patient had one or both of the C677T and A1298C polymorphisms. In addition, tests were performed to determine the levels of folate and vitamin B12 in the body. The patient was identified as having normal folate and B12 levels. The patient was placed on a regimen of 3 mg L-methylfolate, 2 mg methyl-B12, and 35 mg vitamin B6 twice per day. He was reexamined 60 days later and reported improvement of all symptoms. He was again reexamined six months later and reported normalization of all symptoms.

### Example 26

Patient 26 was an 8-year-old child with a history of inability to sleep, inability to concentrate, and blurry and reduced vision. Through genetic testing, it was determined that the patient had one or both of the C677T and A1298C polymorphisms. In addition, tests were performed to determine the levels of folate and vitamin B12 in the body. The patient was identified as having normal folate and B12 levels. The patient was placed on a regimen of 3 mg L-methylfolate, 2 mg methyl-B12 and 35 mg vitamin B6 once per day. The child was reexamined 90 days later and reported improvement of all symptoms. The child was again reexamined six months later and reported normalization of all symptoms.

### Example 27

Patient 27 was a 46-year-old male athlete that presented with muscle soreness, cramping, and fatigue lasting three to four days following free weight workouts and/or participation in any energetic sports such as basketball. The patient reported suffering a career ending back injury during a prior college football event. Through genetic testing, it was determined that the patient had one or both of the C677T and A1298C polymorphisms. The patient was placed on a regimen of 3 mg L-methylfolate, 2 mg methyl-B12 and 35 mg vitamin B6 twice per day. He was reexamined four months later and reported that the time to full muscle recovery after free weight workouts and high output sports was reduced to one day.

### Example 28

Patient 28 was 45-year-old male competitive athlete that presented with muscle soreness, cramping, and fatigue lasting four to five days following participation in competitive running, cycling, and swimming events. Through genetic testing, it was determined that the patient had one or both of the C677T and A1298C polymorphisms. The patient was placed on a regimen of 3 mg L-methylfolate, 2 mg methyl-B12 and 35 mg vitamin B6 twice per day. He was reexamined four months later and reported that the time to full muscle recovery after running, biking, and/or swimming was reduced to two to three days.

What is claimed is:

1. A method of preventing, treating, or otherwise improving one or more disease states related to a metabolic disorder of the folate cycle or BH4 cycle, the method comprising:
a) identifying a subject organism having one or more metabolic disorders of the folate cycle or BH4 cycle, and
b) administering to the subject organism an effective amount of one or more downstream folate compounds and, optionally, methyl-B12.

2. The method of claim 1, wherein said one or more disease states is selected from the group consisting of an abnormal blood lipid profile; fibromyalgia; erectile dysfunction; diseases associated with HLA-DQ gene alleles DQA1*03, DQA1*05, DQA1*0501, DQA1*0505, DQB1*02, DQB1*0201, DQB1*0202, DQB1*0301, DQB1*0302, DQB1*0303, and DQB1*05; headaches and/or migraines; muscle soreness, cramping, fatigue, and/or recovery and repair in athletes; and idiopathic neuropathy, inability to sleep, inability to concentrate, and/or neurological development issues in children.

3. The method of claim 2, wherein the subject organism is selected from the group consisting of a child, a diabetic patient, a bariatric patient, a cardiovascular patient, a post surgical patient, and an athlete.

4. The method of claim 3, wherein the method of administration is selected from the group consisting of an energy drink and a drink designed for diabetics.

5. The method of claim 1, wherein the subject organism is not folic acid deficient.

6. The method of claim 5, further comprising c) decreasing the subject organism's intake of folic acid.

7. The method of claim 6, wherein the subject organism is a human.

8. The method of claim 7, wherein the one or more downstream folate compounds are selected from the group consisting of DHF, THF, 5FITHF, 5,10-METHF, 5MTHF, and L-methylfolate.

9. The method of claim 8, wherein the one or more downstream folate compounds comprise L-methylfolate.

10. The method of claim 9, wherein the L-methylfolate is provided in a dose of 1 mcg-25 mg/day.

11. The method of claim 9, wherein the L-methylfolate is provided in a dose of 1-25 mg/day.

12. The method of claim 11, wherein the method further involves administering an effective amount of methyl-B12.

13. The method of claim 12, wherein the methyl-B12 is administered in a dose of 1-2.5 mg/day.

14. The method of claim 1, wherein the malfunction in one or more of the folate cycle and BH4 cycle is one or more of the C677T and A1298C genetic polymorphisms.

15. The method of claim 2, wherein the one or more disease states comprises an abnormal blood lipid profile.

16. The method of claim 15, wherein the abnormal blood lipid profile comprises one or more of high total cholesterol, high triglycerides, high LDL, low HDL, or a high LDL to HDL ratio.

17. The method of claim 13, wherein the one or more disease states comprises an abnormal blood lipid profile.

18. The method of claim 17, wherein the abnormal blood lipid profile comprises one or more of high total cholesterol, high triglycerides, high LDL, low HDL, or a high LDL to HDL ratio.

19. The method of claim 2, wherein the one or more disease states comprises a disease associated with one or more of HLA-DQ gene alleles DQA1*03, DQA1*05, DQA1*0501, DQA1*0505, DQB1*02, DQB1*0201, DQB1*0202, DQB1*0301, DQB1*0302, DQB1*0303, or DQB1*05.

20. The method of claim 13, wherein the one or more disease states comprises a disease associated with one or more of HLA-DQ gene alleles DQA1*03, DQA1*05, DQA1*0501, DQA1*0505, DQB1*02, DQB1*0201, DQB1*0202, DQB1*0301, DQB1*0302, DQB1*0303, or DQB1*05.

21. The method of claim 2, wherein the one or more disease states comprises fibromyalgia.

22. The method of claim 13, wherein the one or more disease states comprises fibromyalgia.

23. The method of claim 2, wherein the one or more disease states comprises headaches or migraines.

24. The method of claim 13, wherein the one or more disease states comprises headaches or migraines.

25. The method of claim 2, wherein the one or more disease states comprises erectile dysfunction.

26. The method of claim 13, wherein the one or more disease states comprises erectile dysfunction.

27. The method of claim 2, wherein the one or more disease states comprises idiopathic neuropathy, inability to sleep, inability to concentrate, and/or neurological development issues in children.

28. The method of claim 13, wherein the one or more disease states comprises idiopathic neuropathy, inability to sleep, inability to concentrate, and/or neurological development issues in children.

29. The method of claim 2, wherein the one or more disease states comprises muscle soreness, cramping, fatigue, recovery and/or repair.

30. The method of claim 13, wherein the one or more disease states comprises muscle soreness, cramping, fatigue, recovery and/or repair.
